(19) United States
(12) Patent Application Publication
(10) Pub. No.: US 2014/0234427 A1
(43) Pub. Date: Aug. 21, 2014

(54) USE OF SYNTHETIC JANUS PARTICLES FOR PREVENTING OR REDUCING CRYSTAL GROWTH

(71) Applicant: UNIVERSITY OF WARWICK, Coventry (GB)

(72) Inventors: Matthew Ian Gibson, Coventry (GB); Stefan Antonius Franciscus Bon, Coventry (GB)

(73) Assignee: UNIVERSITY OF WARWICK, Coventry (GB)

(21) Appl. No.: 14/349,329

(22) PCT Filed: Oct. 5, 2012
(86) PCT No.: PCT/GB2012/052465

§ 371 Date: Apr. 2, 2014

(30) Foreign Application Priority Data
Oct. 7, 2011 (GB) 1117304.4

(51) Int. Cl. A01N 1/02; A01G 1/00; C07C 7/20; C09K 8/52; A61K 47/32; A23L 3/375

(52) U.S. Cl. CPC A01N 1/0221; A61K 47/32; A23L 3/375; C07C 7/20; C09K 8/52; A01G 1/001
USPC 424/489; 424/501; 426/327; 426/654; 435/1.3; 435/374; 435/404; 585/2; 252/70; 507/90; 47/58.1 R

(57) ABSTRACT

The invention provides a method of preventing or reducing the growth of crystals in a substance which is susceptible to crystal growth in which colloidal particles having an amphiphilic structure, e.g. Janus particles, are contacted with the substance. Colloidal particles suitable for use in the invention include cross-linked, colloidal materials formed from hydrophobic monomers such as acrylates or methacrylates and hydrophilic monomers such as those derived from acrylic and/or methacrylic acid. The colloidal particles find particular use in methods of cryopreservation of biological samples (e.g. cells, tissues or organs), as a texture modifier in frozen food products, in the inhibition of gas hydrate formation, and as scale inhibitors.

The present invention relates generally to crystal growth inhibiting agents and, more specifically, to the use of amphiphilic colloidal materials in reducing or inhibiting the growth of ice crystals.

The materials herein described have a wide range of industrial, medical and agricultural applications. In particular, these find use in reducing the formation of large ice crystals in frozen foods, as scale inhibitors in the petrochemical industry, and as cryopreservation agents in minimising structural damage of biological materials such as cells, tissues and organs during freezing and subsequent thawing.

Anti-freeze proteins (AFPs) which protect organisms during exposure to sub-zero temperatures have been isolated from many species, both animal and plant, and allow them to survive in climates which would otherwise lead to freezing and death. (see Harding et al., Eur. J. Biochem. 270:1381-1392, 2003; Harding et al., Eur. J. Biochem. 264: 653-665, 1999; and DeVries et al., Science 7: 1073-1075, 1969). Two unique classes of proteins exist: (i) anti-freeze glycoproteins from polar fish (AFGPs) which are based on a highly conserved and regular tripeptide repeat sequence (Ala-Ala-Thr) with a disaccharide unit on the threonine residue; and (ii) anti-freeze proteins which are found in many unrelated animals, insects and plants and are more structurally diverse in terms of both primary and secondary structures. These proteins display three main macroscopic anti-freeze effects: a non-equilibrium freezing point depression (thermal hysteresis, TH); dynamic ice shaping (DIS); and ice re-crystallisation inhibition (RI).

Previous studies have suggested that anti-freeze proteins may be used in a number of different applications, for example in organ/tissue cryostorage. Cryopreservation using AFPs is, however, complex. Although studies have found that relatively low concentrations of winter flounder (Pseudoplueronectes americanus) AFP enhance the survival of red blood cells cryopreserved in hydroxyethyl starch solutions, at high concentrations this was found to induce additional damage to the cells due to preferential growth of ice around the cells on warming (see Carpenter et al., Proc. Natl. Acad. Sci. 89: 8953-8957, 1992). This damage was attributed to the formation of long thin spicular (i.e. needle-like) ice crystals at higher AFP concentrations. Damage due to the formation of needle-like structures (ice shaping) is associated with freezing point depression properties; growth at the hysteresis freezing point is due to the binding of water molecules to the basal planes of the ice crystals such that these grow like long spears. When testing a number of different types of native AFPs as a cryoprotectant for mouse sperm, these were also found to cause increased damage to the sperm due to the re-crystallisation of extracellular ice on warming. Such effects were observed at all concentrations tested, ranging from 1-100 μg/ml (see Koshimito et al., Cryobiology 45: 49, 1992).

A number of synthetic peptides, designed to function as AFGPs, have been made and tested but found to exhibit the same problem. For example, when used at increased concentrations, these anti-freeze ‘mimics’ were found to reduce the viability of blood and pancreatic islet cells (see Matsumoto et al. Cryobiology 52: 90-98, 2006).

There has also been some suggestion that certain AFGPs, especially when used at higher concentrations, are associated with cytotoxic effects. AFGP8, a short naturally occurring AFGP, has been shown to induce toxicity in human cells (see Liu, Biomacromolecules 8: 1456, 2007).

Ice re-crystallisation in which large ice crystals grow at the expense of smaller ones has been identified as the key cause of cellular damage during cryopreservation of cells and organs and is known as ‘Ostwald ripening’. It is this effect which is also responsible for the poor texture of frozen foods, such as ice-creams and frozen desserts. Previous studies using anti-freeze proteins have focused only on TH and DIS and therefore the key structural features required for RI activity are not fully understood (see Tachibana et al., Angew. Chem. Int. Ed. 43: 856-862, 2004; and Peltier et al., Cryst. Grow. Des. 10: 5066-5077, 2010). Peptide mimics with significantly simplified structures have been shown to maintain RI activity in some cases, but the exact features responsible for this are still not understood (Tam et al., J. Am. Chem. Soc. 130: 17494-17501, 2008).

Despite the obvious potential of AF(G)Ps, their low availability, potential toxicological and immunological issues, and the problems of degradation during storage or sterilisation has so far limited their application and a deeper understanding of their mode of action. Although synthetic AFPs have been proposed, their preparation often involves complex multi-step synthetic steps which does not lend these to commercial applications. These also suffer from some of the same toxicological problems as the native substances.

Thus a need still exists for alternative materials which are capable of inhibiting crystal growth and, in particular, for such materials which may be produced using synthetic routes which can readily be scaled-up to produce these in large amounts and at low cost for commercial use.

What the present inventors have now recognised is that materials which are effective in inhibiting the growth of crystals (i.e. having RI activity) are key to overcoming the limitations of known anti-freeze agents.

Specifically, the inventors have found new crystal growth inhibiting agents which may be used in a wide range of applications where it is important to minimise or prevent crystal growth, for example in the cryopreservation of cells and organs and in improving the texture of frozen foods. These agents comprise colloidal particles having an amphiphilic structure. Their simple structure means that these materials can be prepared using known fabrication routes which are straightforward and which can be scaled-up easily using conventional industrial processes for particle synthesis. Significantly, their mechanism of action does not require precise ‘matching’ of the crystal inhibitor to a specific ice-crystal face which has been indicated to be important for certain AFPs.

As a result of their investigations, the inventors have surprisingly discovered that colloidal particles which are amphiphilic in character are potent inhibitors of ice re-crystallisation. In some cases these have been found to be effective at picomolar concentrations.

Viewed from one aspect the invention thus provides the use of colloidal particles having an amphiphilic structure as a crystal growth inhibiting agent. Methods of preventing or reducing crystal growth in which an effective amount of such particles is contacted with a substance which is susceptible to crystal growth also form an aspect of the invention.

The colloidal particles herein described are particularly effective in preventing or reducing the growth of ice crystals and this forms a preferred aspect of the invention. However, the inventors' findings extend to other types of inorganic and organic crystals whose growth can cause adverse effects. For example, in the oil and gas field, the growth of crystalline hydrates such as clathrates downhole during drilling operations and the formation of scale due to a build-up of mineral deposits (e.g. calcium carbonate) in transport pipes represent significant problems.

By definition, “colloidal particles” have at least one of their dimensions which is about 1 μm or below. Preferably, these will have one or more dimensions which are in the range of 1 nm to 1 μm. More preferably, these will have no dimension which is larger than 1 μm. The use of the term “particle” is intended to refer to solid matter which has a clear phase boundary.

The term “amphiphilic”, when used in relation to the particles herein described, is intended to mean that they have at least one region which is more hydrophobic than the rest of the particle. The particles may have more than one such region. Typically, the particles will have at least one hydrophobic region and at least one hydrophilic region.

The precise nature of the colloidal particles for use in the invention is not limiting; any colloidal particle having the desired amphiphilic character under the conditions in which it is intended to be used may be employed.

Colloidal particles which are amphiphilic are generally known and described in the literature. Such particles are often referred to as “Janus” particles and may vary in shape, for example, from spherical to egg-like (ellipsoid), “snowman” and dumb-bell (peanut-shaped). The precise shape of the particles is not critical to performance of the invention and these may, for example, either possess dual surface functionality or may consist of two or more joined components which have the required hydrophobic/hydrophilic properties. Those particles having one or more ‘lobes’ or ‘protrusions’ which give rise to the desired anisotropy (i.e. which are non-spherical) are generally preferred. Especially preferred are particles which are dumb-bell shaped having two lobes; one which is hydrophobic and one which is hydrophilic. The size of the lobes can vary and these need not be identical in shape and size, i.e. the particle may be non-symmetrical. Variation in the relative size of the lobes alters the hydrophobic/hydrophilic ratio of the particles; the ability to manipulate the relative lobe size enables the properties of the particle to be precisely tuned depending on the desired end use.

The particles for use in the invention will generally have a diameter which is smaller than the length scale of the crystals. Crystal sizes vary depending on the nature of the crystal, but in the case of ice crystals these will generally have a minimum dimension of about 1 μm. Typical particle diameters will thus range up to about 1 μm. Those particles having sub-micron dimensions are, however, generally preferred, and these may range in size from 5 nm to 1 μm, more preferably from 100 nm to 600 nm. Nanoparticulate materials are especially preferred for use in the invention.

Preferred for use according to the invention are colloidal polymer particles, for example, those having an anisotropic surface composition arising from one hydrophilic surface region and one hydrophobic surface region. Such particles and methods for their preparation are known in the art. Anisotropy may arise from the use of comonomers having functional groups which give rise to the desired hydrophilic/hydrophobic character of the polymer material. Alternatively, polymer particles may be suitably functionalised whereby to introduce the required anisotropy using known techniques.

Monomers which may be used in the preparation of the polymeric particles may be readily selected by those skilled in the art.

Hydrophobic monomers useful for forming the polymer materials include vinyl monomers having the formula R¹R²C═CH₂ in which R¹ and R² are organic groups. The hydrophobic monomer can be any acrylate or methacrylate, such as butyl methacrylate, butyl acrylate, 2-ethyl hexyl(meth)acrylate, benzyl meth(acrylate), and their vinyl acetate derivatives (VEOVAs), etc. Of these, meth(acrylates) and especially those having a short chain alkyl group (e.g. C₁₋₆ alkyl) are preferred and include, methyl methacrylate, ethyl methacrylate, propyl methacrylate, iso-propyl methacrylate, butyl methacrylate and isobutyl methacrylate. Other suitable hydrophobic monomers include vinyl aromatic monomers such as styrene and substituted styrenes. Unsubstituted styrene is particularly preferred.

Polystyrene is particularly preferred as the hydrophobic component of the polymeric particles.

Hydrophilic monomers for use in the formation of the polymeric materials can be any vinyl monomer having one or more hydrophilic groups. Examples of hydrophilic groups include carboxylic acids, sulfones, sulfonic acids, phosphates and phosphonates, amino groups, alkoxy groups, amide groups, ester groups, acetate groups, poly(ethylene glycol) groups, poly(propylene glycol) groups, hydroxy groups, or any substituent that carries a charge (whether positive or negative). Particularly suitable hydrophilic monomers include those based on acrylic and/or methacrylic acids, such as hydroxyethyl methacrylate (e.g. 2-hydroxyethyl methacrylate), hydroxypropyl methacrylate, methacrylic acid, acrylic acid, PEG-methacrylate, dimethyl aminoethyl methacrylate. Other suitable hydrophilic monomers include vinyl benzyl triethyl ammonium chloride, styrene sulfonate, vinylbenzoic acid, vinyl sulfonic acid, vinyl phosphonate, etc.

A preferred combination of monomers for use in preparing the hydrophilic region of the polymeric particles is styrene sulfonate and PEG-methacrylate.

The polymer materials may optionally be cross-linked with known cross-linking agents such as divinyl benzene, butadiene, isoprene, ethylene glycol, di(meth)acrylate and bisacrylamide.

A preferred method for use in producing the polymeric particles herein described is based on the seeded polymerisation technique. This involves heating of monomer-swollen cross-linked polymer particles whereby to cause elastic stress which results in phase separation and macroscopic deformation of the particles. This provides a convenient way to manipulate the geometry and surface properties of non-spherical particles. More specifically, in a first step, lightly cross-linked seed particles are produced, for example using an emulsion polymerisation method. The use of a hydrophilic comonomer in this first step results in the production of a hydrophilic shell. The resulting particles are then swollen with a hydrophobic monomer in the presence of a polymerisation initiator and, optionally, in the presence of a further cross-linking agent. In a second step, heating and polymerisation produces the hydrophobic lobe. The final particle consists of two lobes: one lobe contains most of the original seed particle and the other lobe mostly contains the newly polymerised material.

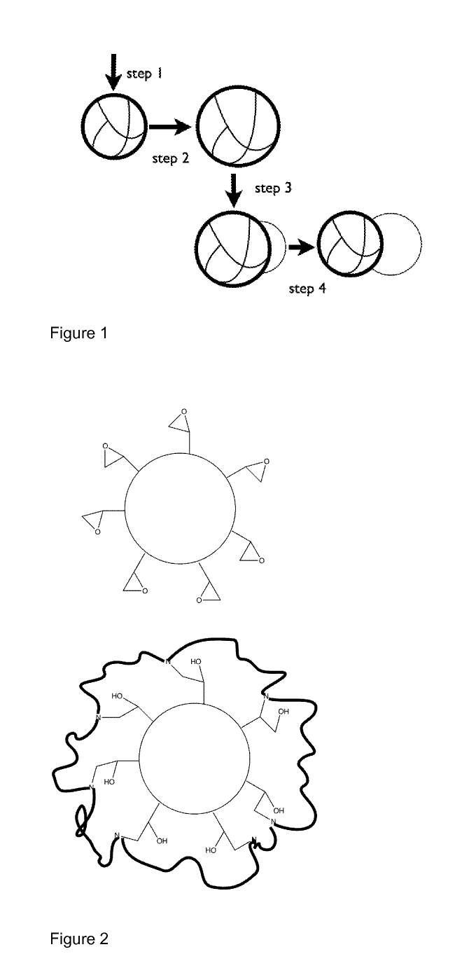

Attached FIG. 1 illustrates an example of a seeded polymerisation method which may be used in preparing a polymeric particle for use in an embodiment of the invention. In step 1 an emulsion polymerisation is carried out to prepare a cross-linked polymer latex (the thick black line indicates the presence of hydrophilic groups at the surface of the particle). In step 2 this seed latex is swollen with a hydrophobic monomer at ambient temperature. In step 3 the swollen latex is heated which causes the system to phase separate driven by entropic contraction of the cross-linked network. In step 4 the system is polymerised to yield the desired amphiphilic anisotropic particle.

In a modification of this method, polymeric particles may be produced having a hydrophobic lobe and reactive sites on the other lobe which are subsequently reacted with the required hydrophilic groups. In this method, the initial cross-linked seed particles are formed using a functional comonomer which provides the desired reactive sites for functionalisation. An example of this process is illustrated in attached FIG. 2 in which the functional comonomer glycidyl methacrylate (GMA) is used to produce the initial cross-linked seed particles. Subsequent reaction of the resulting particles containing GMA with poly(ethylene imine) (PEI) causes the epoxy rings on the surface of the particles to attach to PEI chains thus giving rise to the desired hydrophilic characteristics.

In any of the seeded polymerisation methods herein described, the precise geometry of the particles is tunable by varying the amount of hydrophobic monomer and/or the cross-linking density and hydrophilic nature of the seed particle. This controls the degree of swelling of the seed particle which affects the size of the hydrophobic lobe. In this way, the desired degree of hydrophobic/hydrophilic character of the particles can be precisely controlled depending on the intended use.

The polymeric particles may be produced by seeded polymerisation methods known in the art. Such methods are described in, for example, Kim et al., Adv. Mater. 20: 3239-3243, 2008; Kim et al., Polymer 41: 6181-6188, 2000; Kim et al., J. Am. Chem. Soc. 128: 14374-14377, 2006; Tang et al., Macromolecules 43: 5114-5120, 2010; Shi et al., Colloid Polym. Sci. 281: 331-336, 2003; Sheu et al., J. Polymer Sci. Pol. Chem. 28: 629-651, 1990; Park et al., JACS 132: 5960-5961, 2010; Mock et al., Langmuir 26(17): 13747-13750, 2010; and Mock et al., Langmuir 22: 4037-4043, 2006, the contents of which are hereby incorporated by reference.

Other colloidal particles having the desired amphiphilic structure are equally suitable for use in the invention and are generally known and described in the literature. A wide range of different types of particles may be used, subject to appropriate surface modification to introduce the necessary hydrophobic/hydrophilic character. Examples of other particles which may be surface modified include inorganic materials such as titania, silicates (e.g. silica nanoparticles), metal oxides (e.g. iron oxide, alumina, etc.). Metal particles may also be used, including nanoparticles made of gold, copper, silver, and other metals. Other particulate materials which may be surface-modified include polymeric materials such as those already described.

Both chemical and physicochemical methods may be employed to modify the surface of the particles, for example to introduce materials which have the desired hydrophobic/hydrophilic properties or which may be further modified to give rise to these. Suitable materials for use in modification of the seed particles include polymers such as polystyrene, poly(meth)acrylates, poly(meth)acrylamides, poly(vinylacetates) and VEOVA derivatives as hereinbefore described. One or more metals or their oxides may alternatively be used to selectively coat the particles. Examples of suitable metals include, for example, gold, silver, platinum, copper, aluminium, cobalt, nickel, etc. As noted, where appropriate, such materials may be further functionalised using methods known in the art.

A number of methods are known for use in the production of particles having assymetric surface structures, for example those based on selective surface modification of a particle. Such methods generally include steps in which a portion (or portions) of the surface layer of a particle is masked before carrying out a chemical modification of the unprotected portion of the particle. Partial immersion of one hemisphere of a particle in a protective varnish layer is one such method. The use of solidified emulsions has also been proposed in which inorganic particles such as silica particles are first adsorbed to the liquid-liquid interface of a wax-in-water emulsion. This is subsequently cooled to “lock” the particles at the solidified wax-water interface. The resulting colloidosomes are sufficiently robust to be washed and chemically modified, for example by reaction in solution or in the gas phase (e.g. by vapour phase deposition of suitable reactants). After chemical modification of the exposed side of the particles, the wax can be dissolved away in an organic solvent.

The air-water interface of a Langmuir trough has also been used to carry out regioselective surface modification of colloidal particles. Other methods include the use of planar solid substrates as protecting surfaces onto which particles are placed as a monolayer; the side of the particle that faces the substrate is protected from modification and the other side may be modified, e.g. chemically or physically, by known methods such as sputtering and stamp coating.

Particles having a partial surface coating of at least one metal may also be used to produce amphiphilic particles suitable for use in the invention. For example, filtration over a membrane covered with nanoparticles (e.g. silica or latex nanospheres) may be employed to deposit metal colloids (e.g. gold colloids) onto them. Inorganic particles, such as silica beads, having a metal on one hemisphere or, alternatively, different metals on opposite hemispheres (i.e. capped with different metals) may also be used. Selective modification of the metal (or metals) can result in the formation of the desired amphiphilic character. Possible modifications include chemical adsorption, formation of self-assembled monolayers, covalent coupling and chemical transformation of metals into other materials. For example, these may be transformed into the corresponding metal oxides by exposure of the particles to oxygen plasma.

Colloidal particles derived from the association of two different materials, e.g. a combination of organic and inorganic materials whose surface chemistries differ sufficiently to give rise to an asymmetric character, may be used as amphiphilic particles or as suitable precursors in their preparation. Examples of organic-inorganic colloidal particles include those in which an organic part, such as a polymer, is combined with an inorganic counterpart such as silica, titania or alumina. One example of such a particle is that consisting of a polymer nodule (e.g. polystyrene) attached to an inorganic nanoparticle (e.g. a nanoparticle of silica). Such structures may be produced by methods such as those described in Reculusa et al., Chem. Mater. 17: 3338-3344, 2005, in which an initially symmetrical seed particle (e.g. a silica seed) is modified by a chemical (e.g. covalent grafting) or physiochemical (e.g. adsorption) process in order to give rise to surface nucleation and growth of an organic polymer nodule at the surface of the seed particle.

As will be appreciated, some of the methods described herein may not directly give rise to the amphiphilic character which is necessary for the resulting particles to be used in the invention. However, where appropriate, any of the assymetric structures which are described herein can readily be made amphiphilic by methods generally known in the art, e.g. by selective functionalisation to introduce hydrophobic or hydrophilic groups.

Other methods which may be used to produce colloidal particles for use in the invention thus include regional deposition of chemicals, for example using techniques such as microcontact printing, liquid-liquid interface templating, or vapour (metal) deposition; micro/nanofluidics; and heterocoagulation/self-assembly. In the case of microcontact printing, objects such as for example microspheres, are locally modified (i.e. functionalised or decorated) through contact with a soft stamp soaked in the coating material (see e.g. Kaufmann et al., “Sandwich” Microcontact Printing as a Mild Route towards Monodisperse Janus Particles with Tailored Bifunctionality, Adv. Mater., 23(1): 79-83, 2011). In liquid-liquid interface templating, particles are partially embedded in liquid wax (droplets) using the phenomenon of Pickering stabilization after which the wax is solidified fixing the position of particles. Chemical modification of the exposed surface areas is then carried out (see e.g. Hong et al., “A Simple Method to Produce Janus Colloidal Particles in Large Quantity,” Langmuir 22: 9495, 2006). In vapour metal deposition techniques, metal such as for example gold, is deposited locally onto a monolayer of spherical particles (see e.g. Anker et al., J. Magn. Mater. 293: 655, 2005). In the case of microfluidics, different liquid streams are combined in, for example, a flow focussing device, thereby generating droplets which can have chemical anisotropy. Solidification leads to anisotropic particles (see e.g. Zhihong et al., J. Am. Chem. Soc. 128 (29): 9408-9412, 2006).

The desired crystal growth inhibiting properties of the colloidal particles may be optimised for any particular end use by varying the respective sizes of the hydrophobic and hydrophilic portions (e.g. lobes). In one embodiment it is preferred that the particles should comprise at least 30% (by volume), more preferably at least 35% (by volume), e.g. at least 40% (by volume) of the hydrophobic component. The relative proportions of hydrophobic and hydrophilic components may be determined by methods known in the art such as scanning electron microscopy (SEM).

The particles herein described are capable of inhibiting and/or reducing crystal growth associated with the freezing or supercooling of substances. Under supercooling conditions, a substance is cooled to a temperature below its freezing point but without a change of state (e.g. in the case of a liquid, this does not become solid under supercooling conditions). Accordingly, the materials find use in a wide variety of applications in which it is desirable to prevent or inhibit ice crystal growth or the growth of other crystals. Amongst such other crystals are those formed in gas hydrates.

Suitable concentrations of the particles will vary depending on the use, but can readily be determined by those skilled in the art. Typically, these will be used in a concentration of up to about 50 mg/ml. Preferably, these may be used in a concentration in the range of from about 500 μg/ml to about 50 mg/ml, e.g. from 1 to 10 mg/ml.

One aspect of the invention relates to the use of the materials herein described in methods of cryopreservation. The recrystallisation of ice during the thawing of cryopresevered biological samples (e.g. cells, tissues, organs) has been indicated as a key source of damage, which limits the routine application of cryopreservation. In this aspect of the invention, the colloidal particles may be used on their own to improve cryopreservation or, alternatively, these may be introduced into any liquid which is intended for use in the storage of any human or non-human cell, tissue or organ in the frozen state, for example any vitrification solution commonly used for cells and/or tissues. Use in the short or long-term storage of biological products intended for transplantation, for example in perfusion solutions or dispersions, is a particularly important aspect of the invention whereby such products can be stored with minimum cellular damage arising from ice crystal growth. Although of particular interest in relation to mammalian (e.g. human) cells and tissues, the invention is not limited to these but extends to other cells, e.g. bacterial cells and yeast cells in which it is important to retain cell or tissue viability following a freeze-thaw process.

Methods for the preservation or cryopreservation of a biological material comprising a cell, organ or tissue comprising contacting said material with a crystal growth inhibiting agent as herein described form a further aspect of the invention.

In a further aspect the invention also provides a method of inhibiting ice re-crystallisation on thawing of an organ, tissue or biological sample, said method comprising the step of contacting said organ, tissue or biological sample with a crystal growth inhibiting agent as herein described prior to or during the step of freezing or supercooling. When used in this aspect of the invention, preferred concentrations of the agent may range from 1 to 50 mg/ml, preferably from 1 to 5 mg/ml.

Examples of biological materials which may benefit from the invention include samples containing a suspension of cells, for example, samples comprising whole blood, blood plasma, blood platelets or red blood cells. Samples containing semen, embryos, etc. may also be treated according to the methods herein described. Amongst the organs which may be protected using the methods herein described are heart, liver, kidney, lung, spleen.

Cryopreservation may be carried out using methods generally known in the art when using anti-freeze agents. Where the sample to be preserved consists of cells, the beneficial effect of the crystal growth inhibiting agent is achieved by contacting said cells with the agent during the period of thawing which is when ice re-crystallisation can occur. In the case where the cells are provided in the form of a cell suspension, this is most readily achieved simply by adding the agent to the suspension fluid in which the cells are provided. When the cells are in the form of organs or tissues, these will generally be immersed in a solution of the agent. Where the organs or tissues contain a vascular system, these will be perfused with a solution of the agent using known perfusion methods. Such solutions will generally contain other substances commonly used in perfusion solutions such as sugars and/or salts.

A further area in which the materials herein described find use is in food technology, specifically as texture modifiers for frozen food products. Many frozen food products (including, but not limited to, ice cream, meat and fruit) suffer from the growth of ice during storage which can adversely affect the texture of the product. For example, ice cream with large crystals has a grainy texture which is unappealing, whereas meat and fruit products which have been frozen tend to lose significant volumes of water when defrosted due to ice-induced damage to the structure of the product. Incorporation of the colloidal particles described herein in any of these food products may be beneficial. When used in any food application, biocompatibility of the particles is important, as well as solubility in any solution in which these may be applied to the product or in any formulation in which these may be provided.

In particular, the materials which are described herein may be used to reduce or inhibit ice crystal growth in food products, for example during their production and/or storage in a frozen state (e.g. at a temperature of between −15° C. and −40° C.). Texture and flavour are typically adversely affected due to the formation of large ice crystals during the freeze-thaw cycle which takes place in most home freezers or on long term storage in the frozen state. This ice crystal growth can be minimised or even prevented entirely when using the materials which are herein described. As a result, the texture, taste and useful storage life of frozen food products can be improved.

The particles may be added to any food which is to be frozen until consumption or which may remain frozen during consumption and may either be incorporated throughout the entire product or, alternatively, applied only to the surface of the product which is where ice crystal growth occurs most readily. The crystal growth inhibiting agent may be added during conventional methods of food preparation and may be added prior to, during, or after freezing of the product. If added after freezing, this is done before the product is finally hardened so that the agent may be mixed into the product. For example, this may be incorporated into frozen foods which are intended to be consumed in the frozen state such as ice creams, frozen yoghurts, sorbets, frozen puddings, ice lollies, etc. whereby to improve mouthfeel due to the lack of large crystal formation during preparation and storage. Typically, the agent will be mixed with other ingredients during the manufacture of the products.

Other frozen food products which may benefit from the invention include frozen fruit and vegetables, such as strawberries, raspberries, blueberries, citrus fruits, pineapples, grapes, cherries, plums, peas, carrots, beans, sweetcorn, broccoli, spinach, etc.

Frozen food products which incorporate the materials herein described and which are intended to be consumed in the frozen state and/or stored in the frozen state form a further aspect of the invention. Preferred food products include ice cream and sorbets which will include other ingredients conventionally found in such products, such as fats, oils, sugars, thickeners, stabilisers, emulsifiers, colourings, flavourings and preservatives. In such products, the total amount of the anti-freeze material will typically be at least about 0.01 wt. %, preferably at least 0.1 wt. %, e.g. about 0.5 wt. %. Ideal concentrations can be readily determined by those skilled in the art in the knowledge that this should be used at as low a concentration as possible whilst still having the desired effect of preventing ice re-crystallisation.

The agents herein described also find use in the inhibition of gas hydrate formation, e.g. during drilling for hydrocarbons such as oil and gas. Gas hydrates are crystalline molecular structures which resemble ice and which form when mixtures of water and gas molecules come into contact. Formation of gas hydrates (e.g. clathrates) is a particular problem encountered in gas pipelines which run along the ocean floor as well as in subterranean formations during the production of oil and gas. When used in oil field applications, the crystal growth inhibiting agent will typically be applied downhole either prior to or during drilling and may, for example, be applied in a hydrocarbon fluid. Such fluids containing the crystal growth inhibiting agent form a further aspect of the invention.

Viewed from a further aspect the invention thus provides a hydrocarbon well treatment composition comprising a carrier liquid containing polymeric particles as herein described. Suitable carrier liquids include organic liquids such as a hydrocarbon or mixture of hydrocarbons, typically a C₃ to C₁₅ hydrocarbon or oil, e.g. crude oil. Alternatively, the carrier liquid may be an aqueous liquid.

Methods of inhibiting hydrate (e.g. clathrate) formation in a crude oil or gas product comprising the step of adding a crystal growth inhibiting agent as herein described to said product form a further aspect of the invention.

In carrying out such methods the polymeric particles may be placed down hole before, during and/or after hydrocarbon production has begun (i.e. extraction of oil or gas from the well). Preferably the particles will be placed down hole in the form of a dispersion in a carrier liquid before production has begun, for example in the completion phase of well construction, and may be applied in combination with other agents known and used in treating hydrocarbon wells, such as scale inhibitors, corrosion inhibitors, surfactants, etc.

Other uses of the materials include the protection of crops and plants from climatic freezing conditions in which these may be externally applied to the crops or plants, typically by spraying. They may also be used as an additive to fluids or liquids which are intended for use as a refrigerant.

Almost any material which is exposed to cycles of freeze-thaw shows a decline in performance over time. For example, road surfaces tend to buckle following extended freeze-thaw periods. The build-up of ice on surfaces is also a major problem in the air industry in which aircraft must be treated with conventional anti-freeze (e.g. ethylene glycol) during winter to ensure that all surfaces are free of ice. Any surface or material which is subjected to freezing conditions may also be treated with the crystal growth inhibiting agent whereby to prevent the growth of ice crystals and subsequent damage. In this aspect of the invention, the particles may be used alone, for example by direct application to the surface, or, more preferably, as part of a formulation as an anti-freeze or as a de-ice product. Surfaces which might be treated include those in the transport sector, such as road surfaces, surfaces of aeroplanes and helicopters (e.g. aeroplane wings), rail tracks, etc. Application of the crystal growth inhibiting agent to a road surface is particularly beneficial in preventing any freeze-thaw damage which may be caused by trapped water. For use in this aspect of the invention, it is envisaged that the particles would be applied (e.g. by spraying) in the form of a fluid in which these are dispersed. Aerosol formulations containing the particles form another aspect of the invention.

In surface treatment, the particles may also be incorporated into surface coatings such as paints whereby to improve their sub-zero performance.

Although in any of the applications described above it is expected that the colloidal material will be used as the sole anti-freeze agent, this may nevertheless be used in combination with other known anti-freeze agents, such as ethylene glycol, propylene glycol, glycerol, sodium chloride or methanol, or in combination with any biological anti-freeze such as trehalose, anti-freeze protein or anti-freeze glycoprotein.

The crystal growth inhibiting agents herein described will generally be used in the form of a solution of the particles in a liquid, i.e. a colloidal dispersion. Suitable liquids include aqueous solutions, e.g. water. Depending on their use, such aqueous solutions may further contain other components known in the art for that particular use. In the context of preserving biological cells, tissues and organs, for example, these may also contain salts, ions, sugars or other nutrients known and used for preserving such materials. Electrolyte solutions containing a crystal growth inhibiting agent as herein described form a further aspect of the invention.

Suitable electrolyte solutions include those known in the art, such as Physiological Saline, Ringer's Injection Solution, Alsever's Solution, cell culture medium, etc. The exact choice of electrolyte will be dependent on the nature of the biological material which is to be preserved and can readily be determined by those of skill in the art.

The invention is illustrated further in the following non-limiting examples and in the attached Figures, in which:

FIGS. 1 and 2 are schematic illustrations of seeded polymerisation methods which may be used for the preparation of polymeric particles for use according to certain embodiments of the invention;

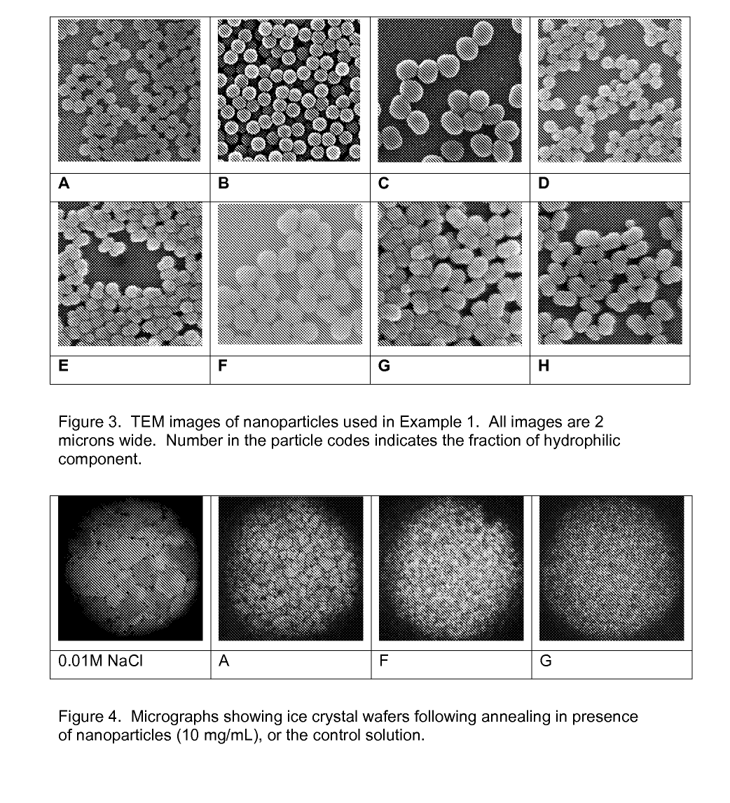

FIG. 3 shows TEM images of nanoparticles produced according to Example 1;

FIG. 4 shows micrographs of ice crystal wafers following annealing in the presence of nanoparticles (10 mg/mL), or a control solution, according to Example 2;

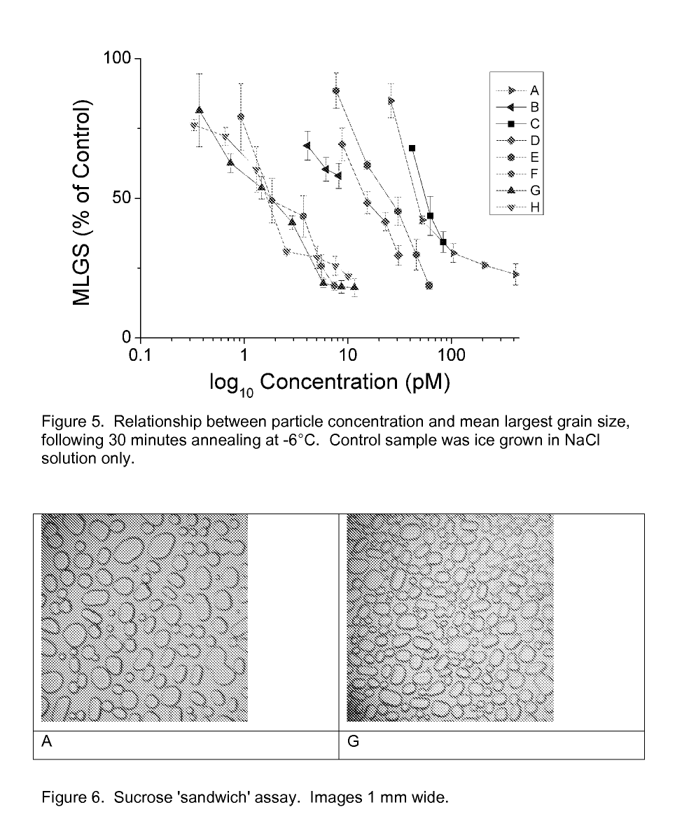

FIG. 5 shows the relationship between particle concentration and mean largest grain size according to Example 2; and

FIG. 6 shows the results from the sucrose ‘sandwich’ assay according to Example 3.

### EXAMPLE 1

### Preparation of Amphiphilic Particles

A two-step emulsion polymerisation process was used to produce dumbbell (peanut-shaped) anisotropic, or ‘Janus’, particles. In the first-step, a lightly cross-linked polymer latex with a hydrophilic shell was made. Styrene sulfonate and a poly(ethyleneglycol)methacrylate-based monomer were used in small quantities as comonomers to provide the hydrophilic surface of the microgel latex particles (ca. 200 nm in diameter). These were subsequently swollen with various amounts of styrene monomer at room temperature. Phase separation, thereby creating the hydrophobic lobe, was induced by entropic contraction of the cross-linked particles upon temperature increase, and promoted further through a second, seeded, polymerisation step initiated by azobisisobutyronitrile (AIBN) to further exclude the introduction of hydrophilic moieties. This second hydrophobic lobe was present in overall particle volume fractions from 0 to 50%.

1.1 Preparation of Hydrophilic Seed Particles (Core Hydrophilic Lobe):

These were made by soap-free emulsion polymerisation. 180 g of distilled degassed water was placed in the reactor and followed by the addition of 20 g of styrene, various amounts of divinyl benzene and 4-styrene sulfonate sodium salt based on the required cross-linked density and colloidal stability. 1.0 g of hydrophilic monomer (in the presence of a small amount of divinyl benzene) was introduced either ab initio or to promote a hydrophilic shell after ca. 50% monomer conversion in 5 mL of water. The polymerization temperature was 70° C. 0.075 g of potassium persulfate was used as initiator.

1.2 Formation of Amphiphilic Particle:

4.0 g of seed latex particles having a total solid content of 1.8% was placed in a glass vial. 0.05 to 0.21 g of a homogenous solution mixture of styrene (6.0 g), divinyl benzene (0.010 g) and AIBN (0.060 g) was added to the latex. The vial was degassed using nitrogen for 10 min and then closed and placed on the oven which had a rotating motor to tumble the sample at a speed of 30 rpm for 24 hours at a temperature of 25° C. After that the oven was heated up to 70° C. for another 24 hours to start the polymerisation after the swelling step. The latex was dialysed against water for one week with daily replacement of the water.

### EXAMPLE 2

### Testing

2.1 Method

The ability of the particles to inhibit the re-crystallisation of ice was measured using a modified ‘splat’ assay which allows quantitative evaluation of the mean largest grain size (MGLS) following annealing of a polycrystalline ice wafer at −6° C. for 30 minutes.

As a reference, a ‘hairy’ particle comprising the same hydrophilic core with grafted poly(styrene sulfonate) polymer chains grown from the surface was also synthesised and tested. The physical properties of this particle and those prepared according to Example 1 are summarised in Table 1, and SEM images showing the peanut-like or dumbbell structure of these particles is shown in FIG. 3.

TABLE 1
Characterisation of nanoparticles

| Code | Hydrophilic (%)^{(a)} | Hydrophobic (%)^{(a)} | Dₕ (nm)^{(b)} | PDI^{(b)} |
|---|---|---|---|---|
| A^{(c)} | 100 | 0 | 178 | 0.027 |
| B^{(d)} | 100 | 0 | 700 | 0.3 |
| C | 95 | 5 | 502 | 0.24 |
| D | 66.23 | 33.7 | 199 | 0.038 |
| E | 58.44 | 41.86 | 490 | 0.1 |
| F | 57.4 | 42.5 | 502 | 0.24 |
| G | 54.21 | 45.78 | 241 | 0.095 |
| H | 52.5 | 47.5 | 240 | 0.044 |

^{(a)}Determined by SEM;
^{(b)}Polydispersity Index determined by DLS;
^{(c)}Seed particle, which forms the hydrophilic component of all other particles;
^{(d)}‘Hairy’ particle with poly(styrenesulphonate) brushes grown from its surface.

2.1.1 Splat Test for Ice Re-Crystallisation Inhibition

A 0.01 M NaCl solution was made using NaCl (Aldrich) and ultra high quality water (UHQ), with 18 MΩ resistively. Ice wafers were annealed on an Otago Nanolitre osmometer (cold stage) fitted onto an Olympus BX41 microscope. A digital camera was attached to the microscope to obtain images (Canon EOS 500 D, 15 megapixels). Images were processed using the manufacturer's software and Image J (Rasband, W. S.; Image J Version 1.37 ed.; National Institutes of Health: Bethesda, Md., USA, 1997-2006). The ‘splat’ assays were conducted according to the method of Knight et al. (Cryobiology, 32: 23, 1995) and described below.

A 10 μL sample of the particle dissolved in 0.01M NaCl solution was dropped 1.5 metres down a hollow tube onto a glass cover slip placed on top of a piece of polished aluminium sat on dry ice (note that NaCl was present to rule out non-specific RI effects). Upon hitting the cover slip, a wafer with diameter of approximately 10 mm was formed instantaneously. The wafer was quickly transferred to the cold stage, and held at −6° C. under nitrogen for 30 minutes. A photograph was taken, through crossed polarisers, of the initial wafer (to ensure that a polycrystalline sample had been obtained), and after 30 minutes through crossed polarisers at a resolution of 2 megapixels. Image J was used to analyse the obtained images. A large number of the ice crystals (30+) were then measured to find the largest grain dimension. The average of this value from 3 individual wafers was calculated to give the mean largest grain size (MLGS), which was expressed as a percentage relative to control ice crystals grown in 0.01 M NaCl.

2.1 Results

FIG. 4 shows the dramatic effect the various nanoparticles have on the ice crystal wafers; particle A (100% hydrophilic) shows no discernable difference from the control ice wafers, but as the hydrophobic fraction is increased the resulting ice crystals are significantly smaller. In the presence of particle G there was no appreciable increase in grain size from the initially nucleated crystals indicating complete arrest of ice re-crystallisation over the time frame studied.

FIG. 5 illustrates the concentration dependence on ice re-crystallisation, showing a clear trend between increasing the size of the hydrophobic lobe and a decrease in ice crystal size. Notably, the most active particles (G and H) were found to halt ice growth at a concentration of ˜5 picomolar. This is remarkably active, even compared to native AFGP 8, which requires micromolar concentrations (i.e. 6 orders of magnitude more).

### EXAMPLE 3

### Testing

3.1 Method

A modified (qualitative) RI assay was also conducted in concentrated sucrose solution. This is more representative of a food science application and has been used to characterise other AFPs. The particles were prepared at 5 mg·mL⁻¹ concentration in a 45 weight % sucrose solution. 5 μL of this solution was placed between two microscope coverslips and rapidly frozen to about −20° C. on the microscope stage. Once frozen (typically less than 30 seconds) the sample was warmed to −6° C. and the temperature maintained for the duration of the experiment. Every 10 minutes a photograph was taken and the particle size (area) was determined using ImageJ software.

3.2 Results

FIG. 6 shows the results of this assay using particles A and G. The sample with particle G clearly has more and smaller ice crystals present, further demonstrating the ability of the particles to inhibit ice re-crystallisation.

1. A method of preventing or reducing the growth of crystals in a substance which is susceptible to crystal growth in which an effective amount of colloidal particles having an amphiphilic structure is contacted with said substance.

2. A method as claimed in claim 1, wherein said crystals are selected from ice crystals, crystalline hydrates and scale.

3. A method as claimed in claim 1 or claim 2, wherein said particles each comprise at least one hydrophobic region and at least one hydrophilic region.

4. A method as claimed in claim 3, wherein the hydrophobic region comprises at least 30% by volume of each particle.

5. A method as claimed in any preceding claim, wherein said particles have a particle diameter in the range of from 1 nm to 1 μm, preferably 5 nm to 1 μm.

6. A method as claimed in any preceding claim, wherein said particles are colloidal polymeric particles, preferably Janus particles.

7. A method as claimed in claim 6, wherein said polymeric particles are formed from hydrophobic monomers having the formula R¹R²C═CH₂ in which R¹ and R² are organic groups.

8. A method as claimed in claim 7, wherein the hydrophobic monomer is an acrylate or methacrylate, or a vinyl aromatic monomer, preferably styrene.

9. A method as claimed in any one of claims 6 to 8, wherein said particles are formed from hydrophilic monomers which comprise a vinyl monomer having one or more hydrophilic groups.

10. A method as claimed in claim 9, wherein the hydrophilic monomers are derived from acrylic and/or methacrylic acid.

11. A method as claimed in claim 10, wherein the hydrophilic monomers comprise styrene sulfonate and PEG-methacrylate.

12. A method as claimed in any one of claims 6 to 11, wherein the polymeric particles are cross-linked, preferably with one or more cross-linking agents selected from divinyl benzene, butadiene, isoprene, ethylene glycol, di(meth)acrylate and bisacrylamide.

13. A method as claimed in any one of claims 1 to 5, wherein said particles are surface-modified inorganic particles, preferably surface-modified silica, alumina or titania particles.

14. A method as claimed in any one of claims 1 to 5, wherein said particles are surface-modified metal particles.

15. The use of colloidal particles as defined in any one of claims 1 to 14 as a crystal growth inhibiting agent.

16. Use as claimed in claim 15 in a method of cryopreservation of a biological sample (e.g. cells, tissues or organs), as a texture modifier in a frozen food product, in the inhibition of gas hydrate formation, or as a scale inhibitor.

17. A method for the preservation or cryopreservation of a biological material comprising a cell, organ or tissue comprising contacting said material with colloidal particles as defined in any one of claims 1 to 14.

18. A method of inhibiting ice re-crystallisation on thawing of an organ, tissue or biological sample, said method comprising the step of contacting said organ, tissue or biological sample with colloidal particles as defined in any one of claims 1 to 14 prior to or during the step of freezing or supercooling.

19. A method of inhibiting hydrate (e.g. clathrate) formation in a crude oil or gas product comprising the step of adding colloidal particles as defined in any one of claims 1 to 14 to said product.

20. A method of protecting crops or plants from climatic freezing conditions, said method comprising externally applying to the crops or plants colloidal particles as defined in any one of claims 1 to 14.

21. A frozen food product (e.g. an ice cream, frozen meat or meat-containing product, or a frozen fruit or vegetable) containing colloidal particles as defined in any one of claims 1 to 14.

22. A hydrocarbon well treatment composition comprising a carrier liquid (e.g. a hydrocarbon or mixture of hydrocarbons, or an aqueous liquid) which contains colloidal particles as defined in any one of claims 1 to 14.

23. An electrolyte solution (e.g. physiological saline, Ringer's injection solution, Alsever's solution, or a cell culture medium) comprising colloidal particles as defined in any one of claims 1 to 14.
